(19) United States
(12) Patent Application Publication
(10) Pub. No.: US 2010/0174275 A1
(43) Pub. Date: Jul. 8, 2010

(54) METHOD AND APPARATUS FOR QUASI-FRACTIONAL INTENSE PULSE LIGHT RESURFACING

(75) Inventor: Gian Franco BERNABEI, Florence (IT)

Correspondence Address: FOLEY AND LARDNER LLP;SUITE 500, 3000 K STREET NW, WASHINGTON, DC, 20007, US

(73) Assignee: MATTIOLI ENGINEERING LTD

(21) Appl. No.: 12/350,466

(22) Filed: Jan. 8, 2009

(51) Int. Cl. A61B 18/18; A61N 5/06

(52) U.S. Cl. 606/9; 607/89

(57) ABSTRACT

An apparatus for treating a skin surface of a patient includes a light source for providing light to the skin surface. The apparatus also includes a shield provided at an output port of the light source so as to be disposed between the skin surface and the light source, the shield including a plurality of holes. The shield is made of a ceramic material that allows light to pass therethrough at an attenuated amount.

### BACKGROUND OF THE INVENTION

1. Field of the Invention

The present invention relates to a method and apparatus for applying to the skin, in a controlled manner, laser light to the skin in order to heat and selectively damage thin superficial layers of the skin, thereby inducing a renewal process of the epidermis.

2. Description of the Related Art

It is well known in the skin treatment art that in order to renew the epidermis layer, induced damage of the skin is required. One such method uses laser radiation that is incident on the skin and that generates several effects on the skin, depending on the wavelength of the laser radiation, the pulse duration of the laser energy applied to the skin, and the radiation energy provided to the skin.

The most commonly used method is CO₂ laser radiation for generating a superficial heating of the skin. When laser light reaches the skin, its intensity decreases exponentially as it progresses down into lower layers of the skin. This means that the thermal energy that is delivered is higher in the first layer and decreases exponentially as its progresses down to lower layers of the skin. Moreover, the first corneum stratus of the skin has a higher absorption than other layers. Such an energy profile is not suitable for a uniform heating of a volume of skin due to the fact that in the superficial (upper) layers, the reached temperature is too high and in the lower layers the reached temperature is not high enough to trigger the desired skin treatment process.

Two principles are used in U.S. Pat. No. 6,518,538, which is incorporated in its entirety herein by reference. First, radio frequency currents are localized in the external layer of the skin due to the skin effect, and thus the heating is localized in a thin (upper) layer of skin.

It is well known that an alternating voltage applied to a conductor generates a current on the external layer of the conductor and the depth depends on the frequency and the resistance of the conductor (so-called skin effect).

Second, the plasma generated at the contact of the skin, due to the radio frequency and a high vacuum generated by a suitable pump, is composed of high energy gas ions that strike the surface of the skin, thereby generating heat in the superficial layer of the skin.

The interaction with the skin has some similarities to the interaction described in U.S. Pat. No. 6,269,271, which is incorporated in its entirety herein by reference.

One advantage of such an approach is by not having electrodes in contact with the skin, a more even distribution of the radio frequency current in the skin is achieved. Also, there is achieved a combined action from the striking gas ions and a more accurate control of the power applied to the skin surface, due to the higher impedance of the plasma that controls the current independently from the electrical conductivity value of the skin.

U.S. Pat. No. 6,518,538 describes an apparatus and a method for skin resurfacing treatment, which provides induced thermal damage of the skin by radio frequency heating and by ion bombardment of the skin.

In U.S. Pat. No. 6,518,538, this dual effect may be achieved by using a pulsed radio frequency generator connected to a probe for coupling to the skin. The probe is preferably made of a non-conductive material (such as glass or plastic), and enables the application of a high vacuum to the skin surface (e.g., 5-10 millibars) over a predetermined (e.g., round) portion of the skin, by using a non-conductive pipe connected to a vacuum pump. At a suitable distance (around 10 millimeters) from the surface of the skin, an electrode (that is housed within the probe) is used to generate a radio frequency field between the electrode itself and the surface of the skin. After reaching a sufficient vacuum (e.g., 5-10 millibars of atmospheric pressure), a high voltage radio frequency electric field is applied between the electrode and the surface of the skin, due to a radio frequency pulse applied to the electrode. Such a radio frequency field triggers a glow discharge inside the probe between the electrode and the skin. A radio frequency current, due to the low impedance of the glow discharge, flows evenly on the surface of the skin, and, due to the skin effect, is limited to the glow discharge area in a depth of about 300 microns. In the surrounding tissues, the current density decreases by the square of the distance from the area covered by the glow discharge within a depth of 300 microns. Moreover, the high energy ions of the glow discharge strike the surface of the skin, thereby providing a plasma skin resurfacing that can be used to remove spider veins, skin brown spots, or port wine stains, for example.

U.S. Pat. No. 6,518,538 describes the providing of a controlled heating of a selected portion of the skin to a depth of about 300 microns. As a result, it is possible to reach a desired temperature of 70 degrees C. or more, which triggers controlled damage to the skin cells to achieve a desired effect. The temperature reached in the described volume of the skin depends primarily on the selected pulse length and the power of the radio frequency generator. Preferably, a temperature reached in the described volume of the skin is a temperature in the range of from 75 degrees C. to 95 degrees C.

U.S. Patent Publication No. 20080021442, which is incorporated in its entirety herein by reference, describes a method and apparatus that use optical radiation to ablate or damage a target area of skin surface for dermatological treatment, which skin surface includes the epidermis and parts of the dermis as the objective or side effect of the desired treatment. In U.S. Patent Publication No. 20080021442, delivery of the electromagnetic radiation to the skin in a predetermined pattern is achieved by either masking parts of the target area of the skin surface in order to protect the masked parts of the skin surface from the electromagnetic radiation, or by utilizing a light beam of relatively small diameter which is scanned across the skin surface by various means in order to generate a specific pattern for affecting superficial thermal skin injury.

The parts of the target area of the skin surface are masked by providing a mask between a light source and the patient's skin, whereby the mask includes many small holes for allowing light to pass from the light source to the patient's skin, and whereby all other portions of the mask that do not have holes entirely block the light from passing through the mask to the patient's skin.

### SUMMARY OF THE INVENTION

The present invention utilizes a method and apparatus for providing light from a light source to skin of a patient to be treated, whereby a ceramic mask that includes a plurality of holes is provided between the light source and the patient's skin, and whereby the ceramic mask allows light to pass through the non-hole portions of the mask at an attenuated rate, such as 70-80%, and whereby light passes through the hole portions of the mask unattenuated. The advantage against the solution of a non transparent shield, is to provide a uniform increase of the temperature on the treated area and a higher increase of the temperature where the holes are located. Instead in the non-transparent shield solution, the increase in the temperature occurs only in the holes. This avoid the delivery of too high energy in the holes for heating the skin and thus limiting the possible damages to the skin that occurs when the energy per square centimeter is too high, i.e. more than 30 Joule/square centimeters. The material that have been found suitable for this application is a Aluminum Oxide with holes laser drilled. The research of the suitable material have been extremely complex. There is the need to avoid increase of the temperature of the shield during the application, but at the same time provide 75% attenuation. The Aluminum Oxide shield reflects 70% of the radiation and transmit the radiation with small attenuation around 5% thus avoiding increase of the temperature of the shield.

### BRIEF DESCRIPTION OF THE DRAWINGS

The invention will become more fully apparent from the following detailed description when read in conjunction with the accompanying drawings with like reference numerals indicating corresponding parts throughout, and wherein:

FIG. 1 shows a device that may be utilized to treat a skin surface in order to provide skin heating, in accordance with a first embodiment of the invention;

FIGS. 2A, 2B and 2C show different view angles of a shield holding unit in accordance with the first embodiment of the invention;

FIGS. 3A and 3B show different view angles of a shield in accordance with the first embodiment of the invention;

### DETAILED DESCRIPTION OF THE PREFERRED EMBODIMENTS

Preferred embodiments of the present invention will be described in detail hereinbelow, with reference to the drawings.

According to the present invention, a skin treatment device is put in contact or adjacent to a patient's skin to be treated, whereby a semi-transparent shield, or mask, is also provided as the distal end of the probe closest to the skin when the skin is treated by the probe. The shield operates to limit the amount of electromagnetic radiation, so as to limit the amount of any damage that may occur to the skin and to apply the electromagnetic radiation with a specific pattern.

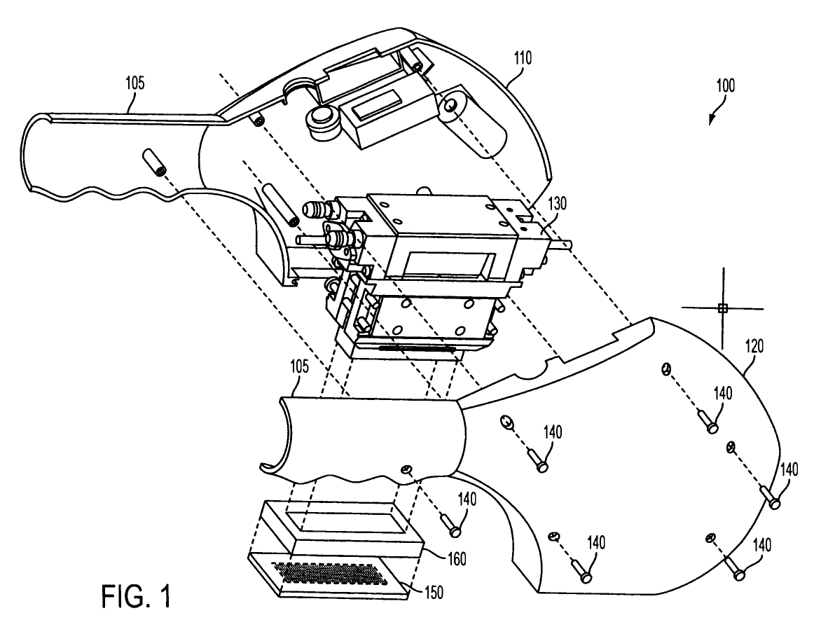

FIG. 1 is a view of the various components making up a skin treatment device 100 according to a first embodiment of the invention. The skin treatment device includes a left-side housing 110 and a right-side housing 120, and a light source 130 disposed within the housings 110, 120. The left and right-side housings 110, 120 are preferably attached to each other, with the light source 130 fixed therebetween, by a plurality of screws 140. A grip portion 105 is provided on the left and right-side housings 110, 120, so that an operator can firmly grasp the skin treatment device 100 so as to treat a particular region of a patient's skin. In the first embodiment, the light source 130 is a Xe lamp light source, which outputs light at an intensity of 150 Joules.

At the bottom of the light source 130, through which light is output towards a patient's skin, a shield 150 and shield holding unit 160 are connected.

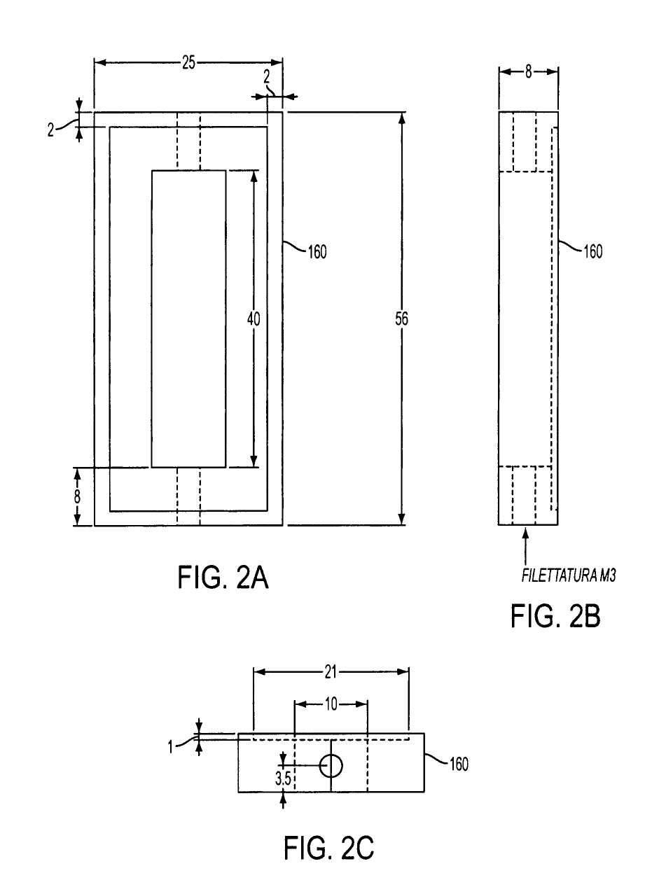

Turning now to FIGS. 2A, 2B and 2C, the shield holding unit 160 is sized to have fitted therein the shield 150, whereby FIG. 2A is a top-down view of the shield holding unit 160, FIG. 2B is a side view of the shield holding unit 160, and FIG. 2C is a front view of the shield holding unit 160. The shield holding unit 160 may be 25 mm by 56 mm in size, with a 21 mm×52 mm opening to thereby fit the shield 150 within the shield holding unit 160.

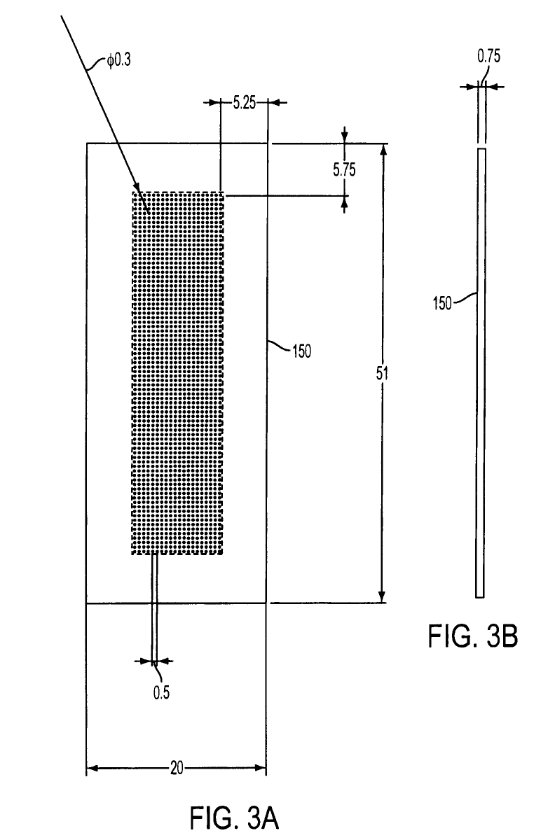

FIG. 3A is a top-down view of the shield 150, and FIG. 3B is a side view of the shield 150. The shield 150 includes a plurality of holes that are provided in a matrix pattern, whereby the holes are 0.5 mm apart from each other in one possible implementation of the shield 150. Other hole spacings are possible while remaining within the spirit and scope of the invention. The shield 150 is sized to fit within the opening of the shield holding unit 160, so that it is held firmly in place within that opening. By way of example, for the 21 mm×52 mm opening of the shield holding unit 160 shown in FIGS. 2A, 2B and 2C, a shield 150 having a 20 mm×51 mm size, such as shown in FIGS. 3A and 3B, is snugly fitted within that opening.

Each of the holes of the shield 150 pass entirely through the shield 150, so as to allow light from the light source 130 to contact the skin of a patient in a non-attenuated manner.

In the first embodiment, the shield 150 is made of a ceramic material, so that the non-hole portions of the shield 150 allow light from the light source 130 to pass through those portions of the shield 150 in an attenuated manner. In one possible implementation, light from the light source 130 passes through the non-hole portions of the shield 150 at 75% of their output light power, thereby resulting in a 25% attenuation due to the ceramic material making up the shield 150 being in the light path to the patient's skin. Other attenuation amounts, such as 15% to 35%, may be utilized while remaining within the spirit and scope of the invention.

By controlling the light amount to be incident on the patient's skin by way of the holes of the shield 150 and by controlling the total amount of light to be incident on the patient's skin by using a shield 150 made of a ceramic material, all portions of the patient's skin are subject to at least some amount of light (and thus are treated), whereby the patient is not subject to too much light output so as to cause damage to the patient's skin. The attenuation amount of the light output from the light source 130 to the patient's skin can be changed by using a thicker or thinner ceramic shield 150, as desired, by easily removing one shield from the shield holding unit 160 and placing another shield into the shield holding unit 160.

In one possible implementation, the ceramic shield 150 according to the first embodiment is made up of the following material: Aluminum Oxide. In another possible implementation, the ceramic shield is constructed from transparent plastic (e.g., polycarbonate), with an optical reflective coating provide thereon, and whereby the holes are provided on the plastic shield by an injection molding process. The optical reflective coating is 75% reflective, and the plastic is 100% transparent, thereby providing a shield having 25% attenuation of light in non-hole portions of the shield (the hole portions of the shield pass through light unattenuated). Of course, to achieve other amounts of attenuation, the optical reflective coating is manufactured to have a particular reflective value (e.g., 70% reflectivity to thereby provide a shield having 30% attenuation of light in non-hole portions of the shield; 80% reflectivity to thereby provide a shield having 20% attenuation of light in non-hole portions of the shield).

In a second embodiment, the light source 130 is a CO₂ laser operated in a fractional mode, i.e., with a matrix of dots, as described in Italian Patent No. 01286634 (F196A118). The CO₂ laser is used to output a single large diameter light beam (non-fractional mode), whereby the combination of the CO₂ laser and the shield provides for a single light beam with a large diameter, which is similar to the output light pattern obtained by using a small diameter laser beam and a wobbling mirror as described in Italian Patent No. 01286634 (F1196A118).

While the present invention has been described with respect to the preferred embodiments, other types of configurations may be possible, while remaining within the spirit and scope of the present invention, as exemplified by the claims.

What is claimed is:

1. An apparatus for treating a skin surface of a patient, comprising:
a light source for providing light to the skin surface;
a shield provided at an output port of the light source so as to be disposed between the skin surface and the light source, the shield including a plurality of holes,
wherein shield is made of a ceramic material that allows light to pass therethrough at an attenuated amount.

2. The apparatus according to claim 1, wherein the ceramic material corresponds to Aluminum Oxide.

3. The apparatus according to claim 1, further comprising:
a housing for holding the light source and the shield in place within the apparatus.

4. The apparatus according to claim 1, wherein the attenuated amount of the light corresponds to 25%.

5. The apparatus according to claim 1, wherein the light source is a laser light source.

6. A method for treating a skin surface, comprising:
providing light to the skin surface; and
providing a shield provided at an output port of the light source so as to be disposed between the skin surface and the light source, the shield including a plurality of holes,
wherein shield is made of a ceramic material that allows light to pass therethrough at an attenuated amount.

7. The method according to claim 6, wherein the ceramic material corresponds to Aluminum Oxide.

8. The method according to claim 6, further comprising:
holding the light source and the shield in place by way of a housing.

9. The method according to claim 6, wherein the attenuated amount of the light corresponds to 25%.

10. The method according to claim 6, wherein the light source is a laser light source.

11. A shield provided at an output port of the light source so as to be disposed between the skin surface and the light source, the shield including a plurality of holes,
wherein shield is made of a ceramic material that allows light to pass therethrough at an attenuated amount.
